(19) United States
(12) Patent Application Publication
(10) Pub. No.: US 2013/0059347 A1
(43) Pub. Date: Mar. 7, 2013

(54) SACCHARIFIED-SOLUTION MANUFACTURING METHOD AND SACCHARIFIED-SOLUTION MANUFACTURING DEVICE USED IN SAID METHOD

(71) Applicants: Tsuyoshi Baba, Saitama (JP); Makoto Uda, Saitama (JP); Tomohiro Imai, Saitama (JP); Shoji Isobe, Saitama (JP)

(72) Inventors: Tsuyoshi Baba, Saitama (JP); Makoto Uda, Saitama (JP); Tomohiro Imai, Saitama (JP); Shoji Isobe, Saitama (JP)

(73) Assignee: HONDA MOTOR CO., LTD., Minato-ku, Tokyo (JP)

(21) Appl. No.: 13/697,334

(22) PCT Filed: May 12, 2011
(86) PCT No.: PCT/JP2011/060976

§ 371 Date: Nov. 9, 2012

(30) Foreign Application Priority Data
May 12, 2010 (JP) 2010-110650

(51) Int. Cl. C12P 19/14; C12M 1/40

(52) U.S. Cl. 435/99; 435/289.1

(57) ABSTRACT

[Problems to be Solved]

Provide is a saccharified-solution manufacturing method and a saccharified-solution manufacturing device, which increases the amount of sugar yielded as a saccharified-solution, when lignocellulose-based biomass is subjected to saccharifying enzyme treatment.

[Solution]

A substrate and ammonia water are mixed at a predetermined mass ratio to yield a substrate mixture, the mixture is retained at a predetermined temperature for a predetermined time period for dissociating lignin from the substrate or swelling the substrate to yield a pretreated ammonia-containing product for saccharification. To a pretreated product for saccharification, which is yielded by separating ammonia from the pretreated ammonia-containing product for saccharification, an acid is added for pH adjustment, and also a saccharifying enzyme is added to yield a substrate/saccharifying enzyme mixture liquid containing a substrate of 15 to 30% by mass. The substrate/saccharifying enzyme mixture liquid is saccharified enzymatically to yield a saccharified-solution.

### TECHNICAL FIELD

The present invention relates to a saccharified-solution manufacturing method and a saccharified-solution manufacturing device used in said method.

### BACKGROUND ART

From a viewpoint of prevention of global warming, a reduction in carbon dioxide emission which is believed to be one of the causes thereof has been required recently. To this end, use of a blend fuel of a liquid hydrocarbon such as gasoline and ethanol for an automobile fuel has been studied.

As such ethanol, those produced by fermentation of a substrate, in which plant substances, e.g. farm products, such as sugarcane and corn are used as the substrate, can be used. Since plants themselves, which are source materials of the plant substances, have absorbed carbon dioxide by photosynthesis, when ethanol originated from the plant substances are burned, the amount of emitted carbon dioxide is equal to the amount of carbon dioxide having been absorbed by the plants themselves. In other words, the so-called carbon-neutral effect can be obtained, such that the overall amount of carbon dioxide emission becomes zero in theory.

On the other hand, there is a drawback that large scale consumption of the sugarcane or corn as a source material for ethanol would reduce the amount of food supply.

Consequently, a technique for producing ethanol using nonfood lignocellulose-based biomass as the plant substances to be used as a substrate, instead of sugarcane, corn, etc. has been studied. Since the lignocellulose-based biomass contains cellulose, ethanol can be yielded by degrading the cellulose by an enzymatic saccharification to a sugar such as glucose, and fermenting the yielded sugar. Examples of the lignocellulose-based biomass include rice straw.

Meanwhile, since the lignocellulose includes as major constituents hemicellulose and lignin in addition to cellulose and the cellulose and the hemicellulose are normally bound tightly to the lignin, an enzymatic saccharification reaction with the cellulose is inhibited as it is. Consequently, for an enzymatic saccharification reaction of the lignocellulose as a substrate, it is desirable to dissociate lignin from the substrate in advance or have the substrate swollen, so that the enzyme should be able to contact the substrate.

In this regard, the term “dissociate” means herein that at least a part of the bonds between lignin and cellulose or hemicellulose as a substrate is broken. The term “swell” means that crystalline cellulose expands due to infiltration of a liquid, which generates gaps in cellulose or hemicellulose as a substrate constituting the crystalline cellulose, or gaps inside a cellulose fiber as a substrate.

For this purpose, a lignocellulose-based biomass saccharification pretreatment device, in which a lignocellulose-based biomass as a substrate is mixed with liquid ammonia to prepare a substrate mixture, and the pressure thereof is decreased rapidly to remove lignin physically from the lignocellulose-based biomass, has been heretofore known (see Patent Literature 1).

In the conventional lignocellulose-based biomass saccharification pretreatment device, a substrate mixture yielded by adding liquid ammonia to the lignocellulose-based biomass is heated and at the same time compressed under pressure so as to prevent ammonia from vaporization. Then, the substrate mixture is discharged from the device.

On this occasion, the substrate mixture is depressurized rapidly following the discharge, and therefore the liquid ammonia vaporizes and the generated ammonia gas expands explosively. As a result, the lignocellulose-based biomass is also expanded rapidly to break a bond between the lignocellulose-based biomass and lignin physically and to yield a pretreated product for saccharification from which lignin has been removed.

In producing ethanol from the pretreated product for saccharification, first a saccharifying enzyme is added to the pretreated product for saccharification to prepare a substrate/saccharifying enzyme mixture liquid to degrade cellulose and hemicellulose contained in the lignocellulose-based biomass as a substrate by the action of the saccharifying enzyme. Examples of the saccharifying enzyme to be used include those produced by a microorganism belonging to the genus Acremonium or the genus Trichoderma.

Next, from the saccharified product, in which cellulose and hemicellulose are degraded, a biomass residue is removed and a saccharified-solution is recovered. Then, by adding an ethanol fermentation microorganism to the saccharified-solution for ethanol fermentation, an ethanol aqueous solution can be yielded. By subjecting the yielded ethanol aqueous solution to a dehydration treatment, such as distillation, it can be purified finally to an ethanol fuel.

As the saccharified-solution manufacturing method using lignocellulose-based biomass as a substrate, for example, a method of treating a pretreated product for saccharification yielded from a substrate mixture of wastepaper as a substrate and liquid ammonia by adding a saccharifying enzyme produced by an Acremonium cellulolyticus C1 strain has been known (e.g. see Patent Literature 2).

Further, as the saccharified-solution manufacturing method using lignocellulose-based biomass as a substrate, for example, a method of treating a pretreated product for saccharification yielded from a substrate mixture of rice straw as a substrate and liquid ammonia by adding a commercial saccharifying enzyme has been known (e.g. see Patent Literature 3).

### CITATION LIST

### Patent Literature

Patent Literature 1: Japanese Patent Laid-Open No. 2005-232453

Patent Literature 2: Japanese Patent No. 4025848

Patent Literature 3: Japanese Patent Laid-Open No. 2010-35431

### SUMMARY OF INVENTION

### Technical Problem

Meanwhile, when a saccharifying enzyme is added to the pretreated product for saccharification to yield a substrate/saccharifying enzyme mixture liquid and the substrate/saccharifying enzyme mixture liquid undergoes saccharifying enzyme treatment to yield a saccharified-solution, it is preferable that the amount of the sugar to be recovered as the saccharified-solution is as much as possible. If the amount of the sugar to be recovered as the saccharified-solution is made as much as possible, the energy required for ethanol fermentation at a downstream step can be decreased. Consequently, it is desirable that the concentration of the lignocellulose-based biomass as a substrate in the substrate/saccharifying enzyme mixture liquid should be as high as possible.

However, there is a drawback that when the concentration of the lignocellulose-based biomass as a substrate in the substrate/saccharifying enzyme mixture liquid is simply increased, the amount of a sugar to be recovered as a saccharified-solution would decrease.

Under such a circumstance, an object of the present invention is to provide a saccharified-solution manufacturing method, which can increase the amount of a sugar to be yielded as the saccharified-solution, when the lignocellulose-based biomass as a substrate is subjected to a saccharifying enzyme treatment.

A further object of the present invention is to provide a saccharified-solution manufacturing device used in the saccharified-solution manufacturing method.

### Solution for Solving Problem

To attain such objects, the present invention is a saccharified-solution manufacturing method, in which a lignocellulose-based biomass as a substrate is pretreated prior to saccharification to yield a pretreated product for saccharification in which lignin is dissociated from the substrate or the substrate is swollen, and thereafter the pretreated product for saccharification is saccharified enzymatically to yield a saccharified-solution, wherein the saccharified-solution manufacturing method comprises: a step of mixing the substrate and ammonia water having a concentration in the range of 20 to 30% by mass at a mass ratio in the range of substrate:ammonia water=1:0.7 to 1:1.3 to yield a substrate mixture; a step of heating and retaining the substrate mixture at a temperature in the range of 25 to 100° C. for a time period in the range of 1 to 100 hours to dissociate lignin from the substrate or to swell the substrate to yield a pretreated ammonia-containing product for saccharification; a step of separating ammonia from the pretreated ammonia-containing product for saccharification to yield a pretreated product for saccharification; a step of adding at least one acid of phosphoric acid, nitric acid and sulfuric acid to the pretreated product for saccharification after the ammonia separation to adjust the pH of the pretreated product for saccharification in the range of 3 to 7 and adding a saccharifying enzyme to yield a substrate/saccharifying enzyme mixture liquid containing the substrate in the range of 15 to 30% by mass with respect to the total mass; a step of saccharifying enzymatically the substrate/saccharifying enzyme mixture liquid to make it into a flowable state to yield a first saccharification treatment product; a step of transferring the first saccharification treatment product to a next step without contacting the outside air; and a step of saccharifying enzymatically the first saccharification treatment product to yield a saccharified-solution as a second saccharification treatment product.

According to the saccharified-solution manufacturing method of the present invention, lignocellulose-based biomass as a substrate and ammonia water are first mixed to yield a substrate mixture.

If liquid ammonia is used as in a conventional saccharified-solution manufacturing method to yield the substrate mixture, there is a drawback that ammonia gas separated from a substrate mixture must be pressurized to approx. 2 MPa to be liquefied for reuse as liquid ammonia, which increases the cost.

According to the saccharified-solution manufacturing method of the present invention, ammonia water is employed instead of liquid ammonia to overcome the drawback. The ammonia water can be recovered at a normal pressure, and therefore reused more easily than ammonia.

Therefore, according to the saccharified-solution manufacturing method of the present invention, a substrate mixture is yielded by mixing lignocellulose-based biomass as a substrate and ammonia water having a concentration in the range of 20 to 30% by mass at a mass ratio in the range of substrate:ammonia water=1:0.7 to 1:1.3. In the substrate mixture, the lignocellulose-based biomass is dispersed in the ammonia water, and further the ammonia water is impregnated uniformly in the lignocellulose-based biomass.

Next, according to the saccharified-solution manufacturing method of the present invention, the substrate mixture is heated to dissociate lignin from the substrate or swell the substrate to yield a pretreated ammonia-containing product for saccharification. By heating the substrate mixture, at least a part of bonds between cellulose or hemicellulose and lignin is broken chemically causing dissociation. Otherwise, in the lignocellulose-based biomass, by infiltration of ammonia water gaps are generated inside cellulose or hemicellulose constituting crystalline cellulose, or inside a cellulose fiber to expand the crystalline cellulose causing swelling.

If the concentration of the ammonia water is less than 20% by mass, dissociation of lignin from the substrate or swelling of the substrate may become insufficient. On the other hand, even if the concentration of the ammonia water exceeds 30% by mass, no additional effect can be obtained with respect to dissociation of lignin from the substrate or swelling of the substrate.

If the ammonia water added to 1 part by mass of lignocellulose-based biomass is less than 0.7 parts by mass, the ammonia water is insufficient, and the ammonia water cannot be impregnated in the substrate uniformly. As a result, dissociation of lignin from the substrate or swelling of the substrate becomes insufficient.

On the other hand, even if the amount of the ammonia water added to 1 part by mass of the substrate exceeds 1.3 parts by mass, no additional effect can be obtained with respect to dissociation of lignin from the substrate or swelling of the substrate. Further, if the amount of the ammonia water added to 1 part by mass of the substrate exceeds 1.3 parts by mass, the energy required for heating the substrate mixture becomes excessive.

Heating of the substrate mixture is carried out by retaining the same at a temperature in the range of 25 to 100° C. for a time period in the range of 1 to 100 hours. As a result, lignin can be dissociated from the substrate adequately, or the substrate can be swollen adequately.

If the temperature during the heating is less than 25° C., the substrate must be retained at the temperature for a time period exceeding 100 hours in order to dissociate lignin from the substrate or swell the substrate, and the energy required for dissociating lignin from the substrate or swelling the substrate becomes excessive. Meanwhile, if the temperature during the heating exceeds 100° C., the time period for retaining the substrate at the temperature in order to dissociate lignin from the substrate or swell the substrate is less than 1 hour, and the control of the retention time becomes difficult. If the temperature during the heating exceeds 100° C., and the retention time exceeds an appropriate value, inconvenience may be caused such that the substrate contained in the substrate mixture may stick by heat to each other partly, or stick by heat to a reactor.

Next, according to the saccharified-solution manufacturing method of the present invention, ammonia is separated from the pretreated ammonia-containing product for saccharification to yield a pretreated product for saccharification, then at least one acid of phosphoric acid, nitric acid and sulfuric acid is added to the pretreated product for saccharification from which ammonia has been separated to adjust the pH of the pretreated product for saccharification in the range of 3 to 7 and also a saccharifying enzyme is added to yield a substrate/saccharifying enzyme mixture liquid containing the substrate in the range of 15 to 30% by mass with respect to the total mass.

The saccharifying enzyme can perform saccharification in the substrate/saccharifying enzyme mixture liquid in the above pH range. Therefore, a saccharified-solution can be yielded by saccharification treatment in the substrate/saccharifying enzyme mixture liquid with the saccharifying enzyme.

According to the saccharified-solution manufacturing method of the present invention, by making the concentration of the substrate contained in the substrate/saccharifying enzyme mixture liquid in the range of 15 to 30% by mass, a larger amount of sugar as the saccharified-solution can be yielded, when the substrate/saccharifying enzyme mixture liquid is subjected to the saccharifying enzyme treatment.

If the concentration of the substrate in the substrate/saccharifying enzyme mixture liquid is less than 15% by mass, the substrate is in small quantity, and the amount of sugar itself to be yielded by the saccharifying enzyme treatment is small, and sugar yielded as the saccharified-solution decreases in quantity.

On the other hand, if the concentration of the substrate exceeds 30% by mass, as a result of the saccharifying enzyme treatment a biomass residue generated from the substrate increases, and the amount of sugar adsorbed on the residue and lost increases. Consequently, sugar yielded as the saccharified-solution decreases in quantity.

Meanwhile, the pretreated product for saccharification from which the ammonia has been separated is substantially lacking in flowability, and therefore it is difficult to be transferred as it is to a step of enzymatic saccharification treatment. Further, if the pretreated product for saccharification is placed in a transportation container, etc. and transferred to a step of enzymatic saccharification treatment, it may be contaminated by various bacteria during the transfer due to contact with the outside air. If contaminated by various bacteria, the produced sugar is consumed by the various bacteria, when the pretreated product for saccharification is saccharified enzymatically, and sugar yielded as the saccharified-solution decreases in quantity.

Consequently, the saccharified-solution manufacturing method of the present invention preferably comprises a step of saccharifying enzymatically the substrate/saccharifying enzyme mixture liquid into a flowable state to yield a first saccharification treatment product, a step of transferring the first saccharification treatment product to a next step without contacting the outside air, and a step of yielding a saccharified-solution as a second saccharification treatment product by saccharifying enzymatically the first saccharification treatment product.

The first saccharification treatment product saccharified into a flowable state as described above can be transferred easily by a pump, etc. to a next step. Therefore, by transferring the first saccharification treatment product saccharified into a flowable state to the next step without contacting the outside air, the contamination by various bacteria can be prevented. As a result, consumption by various bacteria of sugar produced during further enzymatic saccharification treatment of the first saccharification treatment product can be suppressed, and a larger amount of sugar can be yielded as a saccharified-solution.

With respect to a step of saccharifying enzymatically the substrate/saccharifying enzyme mixture liquid into a flowable state to yield a first saccharification treatment product, and a step of saccharifying enzymatically the first saccharification treatment product to yield a saccharified-solution as a second saccharification treatment product, the conditions for enzymatic treatments are different. Namely, for making the substrate/saccharifying enzyme mixture liquid to a flowable state, a treatment under severer conditions is required, but once a flowable state is attained, a treatment under milder conditions may be conducted.

Consequently, by dividing the saccharifying enzyme treatment into a step of saccharifying enzymatically the substrate/saccharifying enzyme mixture liquid into a flowable state to yield a first saccharification treatment product, and a step of saccharifying enzymatically the first saccharification treatment product to yield a saccharified-solution as a second saccharification treatment product, the treatment can be conducted efficiently.

According to the saccharified-solution manufacturing method of the present invention, it is preferable that both the substrate/saccharifying enzyme mixture liquid and the first saccharification treatment product saccharified into a flowable state are saccharified by an enzyme for degrading cellulose and hemicellulose. By doing so, in any of the substrate/saccharifying enzyme mixture liquid and the first saccharification treatment product, sugar can be yielded from both cellulose and hemicellulose, and a larger amount of sugar as the saccharified-solution can be yielded.

Further, according to the saccharified-solution manufacturing method of the present invention, it is preferable that the substrate/saccharifying enzyme mixture liquid is saccharified into a flowable state, and, when the viscosity of the yielded first saccharification treatment product reaches a range of 30 to 1000 Pa·s, it should be transferred to a step of yielding a saccharified-solution.

If the viscosity of the first saccharification treatment product exceeds 30 to 1000 Pa·s, transfer by a pump, etc. may become difficult. In order to make the viscosity of the first saccharification treatment product less than 30 Pa·s, it must be retained at a predetermined temperature for a prolonged time period, and the manufacturing cost reduction becomes difficult.

The saccharified-solution manufacturing method of the present invention can be carried out advantageously by use of a saccharified-solution manufacturing device according to the present invention.

A saccharified-solution manufacturing device according to the present invention is a saccharified-solution manufacturing device, in which lignocellulose-based biomass as a substrate is pretreated prior to saccharification to yield to yield a pretreated product for saccharification in which lignin is dissociated from the substrate or the substrate is swollen, and thereafter the pretreated product for saccharification is saccharified enzymatically to yield a saccharified-solution, wherein the saccharified-solution manufacturing device comprises: a first saccharification treatment unit configured to mix the substrate and ammonia water having a concentration in the range of 20 to 30% by mass at a mass ratio in the range of substrate:ammonia water=1:0.7 to 1:1.3 to yield a substrate mixture by; to heat and retain the yielded substrate mixture at a temperature in the range of 25 to 100° C. for a time period in the range of 1 to 100 hours to dissociate lignin from the substrate or to swell the substrate to yield a pretreated ammonia-containing product for saccharification; to separate ammonia from the yielded pretreated ammonia-containing product for saccharification to yield a pretreated product for saccharification; to adjust the pH of the pretreated product for saccharification after the ammonia separation in the range of 3 to 7 and add a saccharifying enzyme to yield a substrate/saccharifying enzyme mixture liquid containing the substrate in the range of 15 to 30% by mass with respect to the total mass; and to saccharify enzymatically the substrate/saccharifying enzyme mixture liquid into a flowable state to yield a first saccharification treatment product; an ammonia water supply unit configured to supply the ammonia water to the first saccharification treatment unit; a pH adjustment unit configured to add at least one acid of phosphoric acid, nitric acid and sulfuric acid to the first saccharification treatment unit to adjust the pH of the pretreated product for saccharification in the above range; a saccharifying enzyme addition unit configured to add a saccharifying enzyme to the first saccharification treatment unit; a transfer unit configured to transfer the first saccharification treatment product without contacting the outside air; and a second saccharification treatment unit configured to perform an enzymatic saccharification treatment of the first saccharification treatment product transferred by the transfer unit to yield a saccharified-solution as a second saccharification treatment product.

### BRIEF DESCRIPTION OF DRAWINGS

FIG. 1 is a system block diagram showing a constitutional example of a saccharified-solution manufacturing device according to the present invention.

FIG. 2 is a graph showing the relationship between the concentration of ammonia water added to lignocellulose-based biomass for a substrate mixture and the saccharification rate by enzymatic saccharification.

FIG. 3 is a graph showing the relationship between the mass of ammonia water added to 1 part by mass of lignocellulose-based biomass for a substrate mixture and the saccharification rate by enzymatic saccharification.

FIG. 4 is a graph showing the relationship between the retention time of a substrate mixture at the respective heating temperatures of 80° C., 100° C., and 120° C. for yielding a pretreated ammonia-containing product for saccharification and the saccharification rate by enzymatic saccharification.

FIG. 5 is a graph showing the relationship between the retention time of a substrate mixture at the respective heating temperatures of 25° C., 50° C., 60° C., 80° C., and 100° C. for yielding a pretreated ammonia-containing product for saccharification and the saccharification rate by enzymatic saccharification.

FIG. 6 is a graph showing the relationship between the pH of a substrate/saccharifying enzyme mixture liquid and the concentration of a yielded saccharified-solution.

FIG. 7 is a graph showing a comparison of the concentrations of saccharified-solutions yielded without contacting the outside air or under a condition allowing contact with the outside air when a first saccharification treatment product is transferred to the next step.

FIG. 8 is a graph showing the relationship between the substrate content amount in a substrate/saccharifying enzyme mixture liquid and the sugar recovery rate in a yielded saccharified-solution.

### DESCRIPTION OF EMBODIMENTS

An embodiment of the present invention will be described below in more detail referring to the appended drawings.

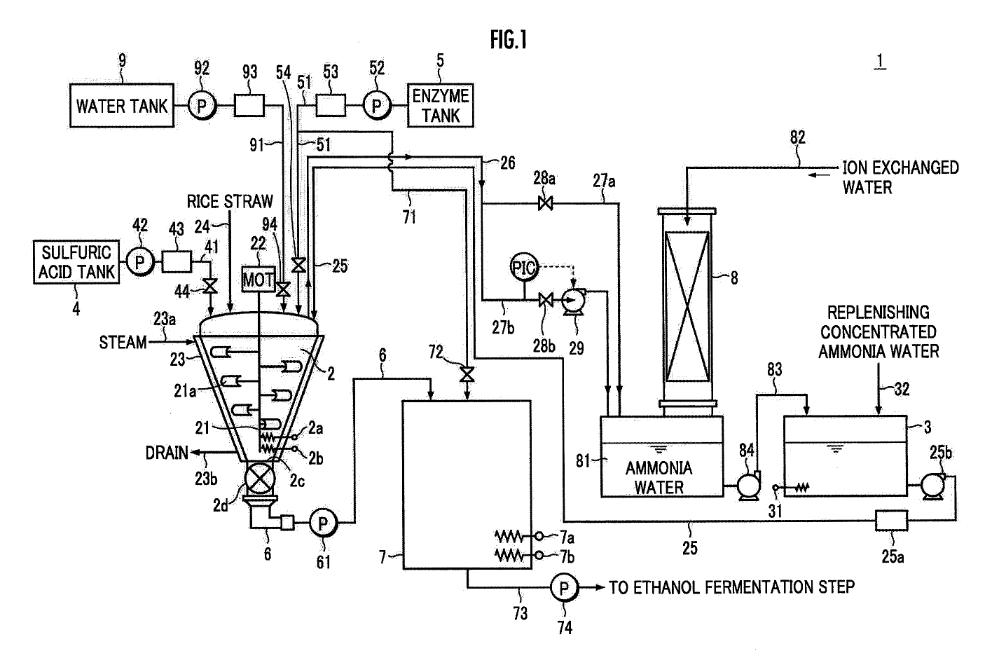

According to a saccharified-solution manufacturing method of the current embodiment a saccharified-solution is manufactured from lignocellulose-based biomass using a saccharified-solution manufacturing device 1 shown in FIG. 1. The constitution of the saccharified-solution manufacturing device 1 will be described below.

The saccharified-solution manufacturing device 1 comprises a reaction vessel 2 as a first saccharification treatment unit, an ammonia water tank 3 as an ammonia water supply unit, a sulfuric acid tank 4 as a pH adjustment unit, an enzyme tank 5 as a saccharifying enzyme addition unit, a transfer line 6 as a transfer unit, and an enzymatic saccharification vessel 7 as a second saccharification treatment unit. The saccharified-solution manufacturing device 1 comprises further an absorber 8 for recovering ammonia separated in the reaction vessel 2, and a water tank 9 for supplying water to the reaction vessel 2.

The reaction vessel 2 is a container formed in an inverse-conical shape, and comprises internally a suspended vertical shaft 21 and in the upper part with a motor 22 for driving rotationally the vertical shaft 21, wherein the vertical shaft 21 is equipped with agitator blades 21a extended in the horizontal direction.

Further, the reaction vessel 2 comprises on the outer side with a jacket 23 for heating the inside or regulating the temperature. The jacket 23 can heat the inside or regulate the temperature of the reaction vessel 2 by feeding steam therein, and is connected in the upper part with a steam supply line 23a for supplying steam and in the lower part with a draining line 23b.

Moreover, the reaction vessel 2 is provided in the upper part with a substrate supply line 24 for supplying lignocellulose-based biomass as a substrate, an ammonia water supply line 25 and an ammonia gas line 26. The ammonia water supply line 25 is connected through a flowmeter 25a and a pump 25b with the ammonia water tank 3, and leads ammonia water supplied from the ammonia water tank 3 to the reaction vessel 2. The ammonia gas line 26 is connected with the absorber 8, and sends ammonia gas generated in the reaction vessel 2 to the absorber 8.

The ammonia gas line 26 branches between the reaction vessel 2 and the absorber 8 into a first exhaust gas line 27a and a second exhaust gas line 27b. The first exhaust gas line 27a comprises an on-off valve 28a halfway. The second exhaust gas line 27b is equipped with an on-off valve 28b halfway and a vacuum pump 29 downstream of the on-off valve 28b.

The absorber 8 is provided with an ammonia water reservoir 81 in the lower part, and with an ion exchanged water supply line 82 in the upper part. The ammonia water reservoir 81 is provided with an ammonia water recycle line 83, and the ammonia water recycle line 83 is connected through a pump 84 with the ammonia water tank 3.

Further, the reaction vessel 2 comprises in the upper part a sulfuric acid line 41 for supplying sulfuric acid from the sulfuric acid tank 4, an enzyme line 51 for adding an enzyme from the enzyme tank 5, and a water line 91 for supplying water from the water tank 9. The sulfuric acid line 41 comprises a pump 42 and a flowmeter 43 downstream of the sulfuric acid tank 4, and connected through an on-off valve 44 with the reaction vessel 2. The enzyme line 51 comprises a pump 52 and a flowmeter 53 downstream of the enzyme tank 5 and connected through an on-off valve 54 with the reaction vessel 2. The water line 91 comprises a pump 92 and a flowmeter 93 downstream of the water tank 9 and connected through an on-off valve 94 with the reaction vessel 2.

The reaction vessel 2 comprises in the lower part with a pH sensor 2a and a temperature sensor 2b, and comprises in the lowest part a discharge port 2c for discharging a first saccharification treatment product. The discharge port 2c comprises an on-off damper 2d which can be freely opened and closed.

The ammonia water tank 3 is connected with the reaction vessel 2 through the ammonia water supply line 25, and also with the ammonia water reservoir 81 of the absorber 8 through the ammonia water recycle line 83. The ammonia water tank 3 comprises an ammonia concentration sensor 31 and a concentrated ammonia water supply line 32 for regulating the concentration of the ammonia water supplied through the ammonia water recycle line 83.

An end of the transfer line 6 is connected through the on-off damper 2d with the discharge port 2c of the reaction vessel 2, and the other end is connected with the upper part of the enzymatic saccharification vessel 7. The transfer line 6 comprises a pump 61 halfway.

The enzymatic saccharification vessel 7, with the upper part of which the transfer line 6 is connected, also comprises an enzyme line 71. The enzyme line 71 branches from the enzyme line 51 downstream of the flowmeter 53 for the enzyme line 51 and is connected through an on-off valve 72 with the enzymatic saccharification vessel 7. The enzymatic saccharification vessel 7 comprises in the lower part a pH sensor 7a and a temperature sensor 7b, and in the lowest part a discharge line 73 for discharging a saccharified-solution as a second saccharification treatment product. The discharge line 73 is connected through a pump 74 with an ethanol fermentation step as the next step (not shown).

Actions of the saccharified-solution manufacturing device 1 according to the current embodiment will be described.

In the saccharified-solution manufacturing device 1 according to the current embodiment, firstly rice straw, namely lignocellulose-based biomass as a substrate is supplied through the substrate supply line 24 to the reaction vessel 2, and at the same time ammonia water is supplied through the ammonia water supply line 25 to the reaction vessel 2. According to the current embodiment, the ammonia water is supplied at a mass ratio in the range of 0.7 to 1.3 parts by mass with respect to 1 part by mass of the rice straw to the reaction vessel 2. The concentration of the ammonia water is 20 to 30% by mass, for example, 25% by mass.

Then, the motor 22 is activated to rotate the agitator blades 21a to stir the rice straw and the ammonia water to yield a substrate mixture in which the rice straw and the ammonia water are mixed. In this connection, all the treatments in the reaction vessel 2 described below are carried out with agitation by means of rotation of the agitator blades 21a.

According to the current embodiment the rice straw as a substrate has been chopped by a cutter mill, such that the cumulative amount of particles with the particle size of 1 mm or larger is 30% by weight or more. Since the rice straw is chopped as described above, the substrate mixture can be yielded without aggregation by stirring the same with ammonia water in the reaction vessel 2 at a low rotation speed for a short time period. If the rice straw is chopped more finely than the above range and stirred with ammonia water, the finely chopped rice straw may aggregate to a pasty state and further stirring may occasionally become difficult.

Then, the substrate mixture in the reaction vessel 2 is heated and retained at a predetermined temperature, for example, in the range of 25 to 100° C., preferably in the range of 60 to 90° C. for a time period in the range of 1 to 100 hours, preferably in the range of 6 to 24 hours. For example, heating of the substrate mixture is carried out by retaining the same at a temperature of 60° C. for 24 hours, or at a temperature of 80° C. for 8 hours. The heating may be carried out by supplying steam into the jacket 23 through the steam supply line 23a, while detecting the temperature of the substrate mixture in the reaction vessel 2 by the temperature sensor 2b.

As a result, a pretreated ammonia-containing product for saccharification can be yielded, in which lignin is dissociated from a substrate composed of cellulose or hemicellulose tightly bound with lignin, or the substrate is swollen. By dissociating lignin from the substrate or swelling the substrate as described above, cellulose or hemicellulose contained in the substrate can be saccharified enzymatically.

When the substrate mixture in the reaction vessel 2 is heated as described above, and the pretreated ammonia-containing product for saccharification is yielded, the inside of the reaction vessel 2 becomes pressurized. Then, if the on-off valve 28a in the first exhaust gas line 27a branched from the ammonia gas line 26 is opened, and the on-off valve 28b in the second exhaust gas line 27b is closed, ammonia gas contained in the pretreated ammonia-containing product for saccharification is emitted spontaneously. As a result, the spontaneously emitted ammonia gas is sent out to the absorber 8 from the ammonia gas line 26 through the first exhaust gas line 27a.

If the ammonia gas is sent out as described above, the pressure inside the reaction vessel 2 decreases with time and the ammonia gas emission rate decreases also. Therefore, when the ammonia gas emission rate decreases below a predetermined value, the on-off valve 28a in the first exhaust gas line 27a is closed, the on-off valve 28b in the second exhaust gas line 27b is opened, and the vacuum pump 29 is activated. By doing so, the ammonia gas can be further sent to the absorber 8 through the second exhaust gas line 27b. As a result a pretreated product for saccharification from which ammonia has been separated can be yielded by emitting adequately the ammonia from the pretreated ammonia-containing product for saccharification.

In this case, if the rice straw as a substrate is chopped as described above, the ammonia can be emitted adequately from the pretreated ammonia-containing product for saccharification, and the amount of ammonia remained in the pretreated product for saccharification can be decreased. However, if the rice straw as a substrate is chopped more finely than the above range and is in a pasty state when mixed with ammonia water, the ammonia remains inside the rice straw in a pasty state and may not be emitted adequately.

As described above, the ammonia gas separated from the pretreated ammonia-containing product for saccharification in the reaction vessel 2 is led to the absorber 8, where it is absorbed by ion exchanged water sprayed from the upper part of the absorber 8 through the ion exchanged water supply line 82 and recovered as ammonia water. The ammonia water recovered as described above is stored in the ammonia water reservoir 81 and recycled to the ammonia water tank 3 through the ammonia water recycle line 83 and the pump 84.

The concentration of the ammonia water recycled to the ammonia tank 3 is regulated to a concentration of 20 to 30% by mass, for example, to 25% by mass in accordance with the ammonia concentration detected by the ammonia concentration sensor 31 using concentrated ammonia water supplied through the concentrated ammonia water supply line 32. The ammonia water regulated to the concentration is supplied to the reaction vessel 2 through the ammonia water supply line 25 and reused for mixing with the substrate.

Then, the pH of the pretreated product for saccharification in the reaction vessel 2 is adjusted to a range of 3 to 7. The pH can be adjusted by opening the on-off valve 44 in the sulfuric acid line 41, and supplying sulfuric acid to the reaction vessel 2 from the sulfuric acid tank 4 by use of the sulfuric acid line 41 and the pump 42, while detecting the pH of the pretreated product for saccharification in the reaction vessel 2 by the pH sensor 2a. In this case instead of detecting the pH of the pretreated product for saccharification in the reaction vessel 2 by the pH sensor 2a, a predetermined quantity of sulfuric acid may be supplied by use of the flowmeter 43. The on-off valve 44 is closed when the supply of sulfuric acid is completed.

Further, in place of sulfuric acid, phosphoric acid or nitric acid may be used, or a mixture of two or more acids out of sulfuric acid, phosphoric acid, and nitric acid may also be used.

Then, a saccharifying enzyme is added to the pretreated product for saccharification in the reaction vessel 2. The saccharifying enzyme can be added by opening the on-off valve 54 in the enzyme line 51 and supplying a predetermined amount of an enzyme aqueous solution to the reaction vessel 2 from the enzyme tank 5 by use of the enzyme line 51 and the pump 52. The amount of the enzyme to be added can be measured by the flowmeter 53.

For the saccharifying enzyme, cellulase, hemicellulase, etc. can be used as an enzyme for degrading cellulose and hemicellulose. Examples of the saccharifying enzyme include GC220 (trade name, manufactured by Genencor Inc.), and Acremonium (trade name, manufactured by Meiji Seika Pharma Co., Ltd.). In this connection the on-off valve 54 is closed when the addition of the enzyme is completed.

By supply of sulfuric acid and addition of a saccharifying enzyme as described above, a substrate/saccharifying enzyme mixture liquid can be yielded, wherein the concentration of the substrate contained in the substrate/saccharifying enzyme mixture liquid can be made in the range of 15 to 30% by mass with respect to the total mass of the substrate/saccharifying enzyme mixture liquid.

With respect to the substrate/saccharifying enzyme mixture liquid, water content may be adjusted according to need by adding water, in order to make the concentration of the substrate in the above range. The water content adjustment may be conducted by opening the on-off valve 94 in the water line 91 and supplying a predetermined quantity of water to the reaction vessel 2 from the water tank 9 by use of the water line 91 and the pump 92. The amount of water to be added can be measured by the flowmeter 93. The on-off valve 94 is closed when the supply of water is completed.

Then, the substrate/saccharifying enzyme mixture liquid is retained and heated in the reaction vessel 2 at a temperature in the range of 25 to 60° C., for example at a temperature of 25° C. for a time period in the range of 5 to 10 hours, for example for 8 hours, or for example at a temperature of 45° C. for a time period in the range of 1 to 4 hours, for example for 2 hours, or for example at a temperature of 60° C. for a time period in the range of 0.3 to 0.8 hours, for example for 0.5 hours. As a consequence a first saccharification treatment product can be yielded, which is the pretreated product for saccharification saccharified enzymatically into a flowable state. There is no particular restriction on the first saccharification treatment product, insofar as it is saccharified enzymatically into a flowable state, and for example it is in a slurry or liquid state having the viscosity in the range of 30 to 1000 mPa·s.

Then, the first saccharification treatment product is transferred to the enzymatic saccharification vessel 7 from the reaction vessel 2 through the transfer line 6 by opening the shut-off damper 2d placed at the discharge port 2c of the reaction vessel 2 and at the same time activating the pump 61 in the transfer line 6. Thus, the first saccharification treatment product can be transferred to the enzymatic saccharification vessel 7 without contacting the outside air by the transfer through the transfer line 6 as described above.

Then, the first saccharification treatment product is further saccharified enzymatically in the enzymatic saccharification vessel 7. For the enzymatic saccharification in the enzymatic saccharification vessel 7, the saccharifying enzyme added to the pretreated product for saccharification in the reaction vessel 2 and carried over from the pretreated product for saccharification and contained in the first saccharification treatment product may be used as it is.

According to need, a predetermined amount of the enzyme may be supplied to the enzymatic saccharification vessel 7 by opening the on-off valve 72 in the enzyme line 71 from the enzyme tank 5 by use of the enzyme lines 51, 71 and the pump 52. The amount of the enzyme to be added can be measured by the flowmeter 53. The on-off valve 72 is closed when the addition of the enzyme is completed.

Then, the first saccharification treatment product is retained and heated in the enzymatic saccharification vessel 7 at a temperature in the range of 30 to 50° C., for example at a temperature of 40° C. for a time period in the range of 80 to 150 hours, for example for 144 hours, or for example at a temperature of 50° C. for a time period in the range of 50 to 150 hours, for example for 72 hours. As a consequence a saccharified-solution as the second saccharification treatment product can be yielded, which is the first saccharification treatment product saccharified enzymatically.

In yielding the second saccharification treatment product, contamination of the first saccharification treatment product by various bacteria can be prevented by transferring the first saccharification treatment product to the enzymatic saccharification vessel 7 without contacting the outside air as described above. As a result, further more sugar can be yielded as the saccharified-solution by suppressing consumption of the produced sugar by various bacteria during the enzymatic saccharification treatment in the enzymatic saccharification vessel 7.

The saccharified-solution is transferred to an ethanol fermentation step through the discharge line 73 and the pump 74, and subjected to ethanol fermentation after removing the biomass residue generated as a result of the saccharification treatment. According to the saccharified-solution manufacturing method of the current embodiment, the concentration of a substrate contained in the substrate/saccharifying enzyme mixture liquid is set at 15 to 30% by mass. Therefore, loss of the produced sugar by adsorption on the biomass residue can be suppressed, and a saccharified-solution with the sugar concentration in the range of 6 to 17% by mass can be subjected to ethanol fermentation

Next, an example of a saccharified-solution manufacturing method using the saccharified-solution manufacturing device 1 shown in FIG. 1 will be described.

According to the current embodiment, the rice straw as a substrate and the ammonia water were supplied to the reaction vessel 2 at a mass ratio of rice straw:ammonia water=1:1 to yield a substrate mixture. The concentration of the ammonia water was varied in the range not higher than 30% by mass.

Then, the substrate mixture was retained and heated in the reaction vessel 2 at a predetermined temperature for a predetermined time period to yield a pretreated ammonia-containing product for saccharification in which lignin is dissociated from the substrate or the substrate is swollen. The temperature was varied in the range of 25 to 120° C., and the time period was varied in the range of 0 to 1000 hours.

Then, a pretreated product for saccharification was yielded by emitting ammonia gas from the pretreated ammonia-containing product for saccharification in the reaction vessel 2. Then the pH was adjusted by supplying sulfuric acid to the reaction vessel 2 from the sulfuric acid tank 4 by use of the sulfuric acid line 41 and the pump 42. Then, the water content was adjusted by supplying water to the reaction vessel 2 from the water tank 9 by use of the water line 91 and the pump 92, and further a substrate/saccharifying enzyme mixture liquid was yielded by supplying a predetermined amount of a saccharifying enzyme to the reaction vessel 2 from the enzyme tank 5 by use of the enzyme line 51 and the pump 52. As the saccharifying enzyme Acremonium (trade name, manufactured by Meiji Seika Pharma Co., Ltd.) was used.

The pH of the substrate/saccharifying enzyme mixture liquid was varied in the range of 3 to 7. Further, concerning substrate/saccharifying enzyme mixture liquid, the content of the substrate was varied in the range of 10 to 35% by mass with respect to the total mass.

Then, by retaining the substrate/saccharifying enzyme mixture liquid in the reaction vessel 2 at a temperature of 45° C. for 2 hours, a first saccharification treatment product enzymatically saccharified into a flowable state was yielded. The viscosity of the first saccharification treatment product was in the range of 30 to 1000 Pa·s.

Then, a saccharified-solution as a second saccharification treatment product was yielded by transferring the first saccharification treatment product to the enzymatic saccharification vessel 7 through the transfer line 6, retaining it at a temperature of 40° C. for 144 hours, and conducting enzymatic saccharification treatment.

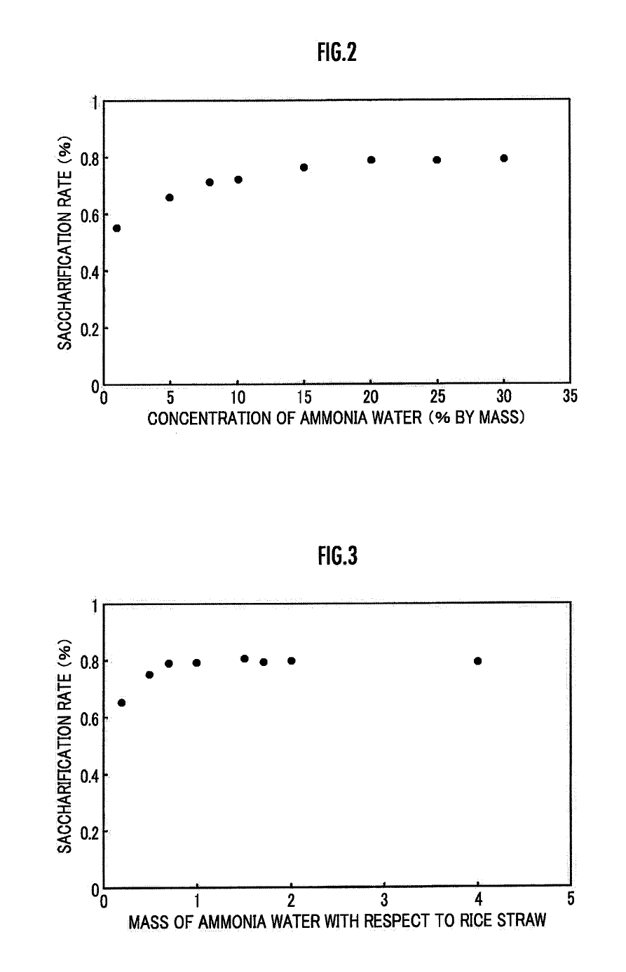

Next, the relationship in the saccharified-solution manufacturing method between the concentration of ammonia water and the saccharification rate of the saccharified-solution is shown in FIG. 2. The saccharification rate is an index of a condition concerning dissociation of lignin from the substrate or swelling of the substrate, and if the saccharification rate is higher, it indicates that dissociation of lignin from the substrate or swelling of the substrate is better.

In FIG. 2, if the concentration of ammonia water is in the range less than 20% by mass, the higher the concentration of the ammonia water is, the higher the saccharification rate becomes; however in the range of 20 to 30% by mass, the saccharification rate substantially levels off. Consequently, it is obvious that lignin can be dissociated adequately from the substrate, or the substrate can be swollen adequately, if the concentration of the ammonia water is set in the range of 20 to 30% by mass.

Next, the relationship in the saccharified-solution manufacturing method between the amount of ammonia water having a concentration of 25% by mass added to 1 part by mass of rice straw as a substrate and the saccharification rate in the saccharified-solution is shown in FIG. 3.

In FIG. 3, if the amount of ammonia water is in the range less than 0.7 parts by mass with respect to 1 part by mass of rice straw, the larger the amount of the ammonia water is, the higher the saccharification rate becomes; however if the ammonia water is in the range not less than 0.7 parts by mass, the saccharification rate substantially levels off. Consequently, it is obvious that lignin can be dissociated adequately from the substrate, or the substrate can be swollen adequately, if the amount of the ammonia water is set in the range of 0.7 to 1.3 parts by mass with respect to 1 part by mass of the rice straw, however no further effect can be obtained, even if it should exceed 1.3 parts by mass.

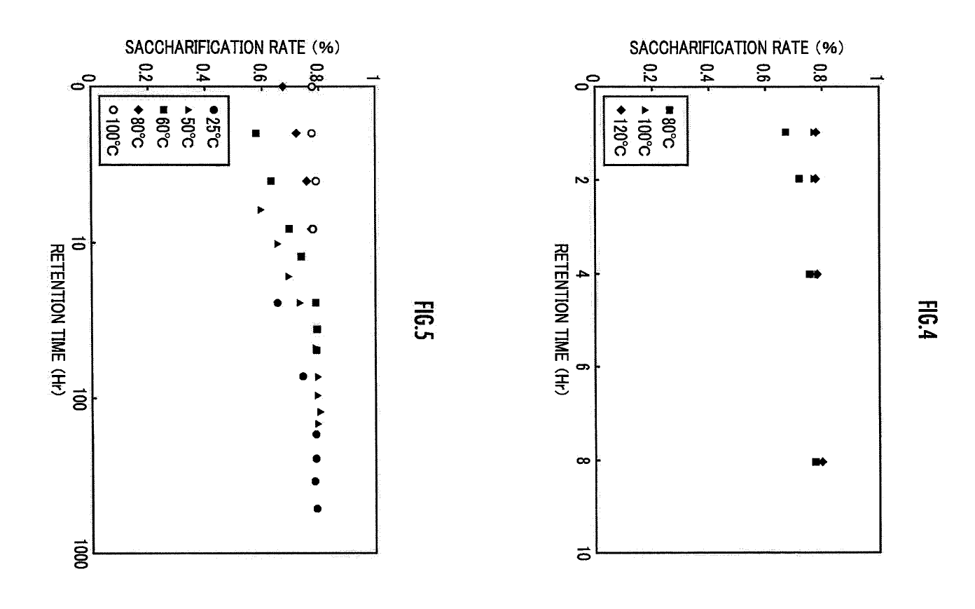

Next, in FIG. 4 and FIG. 5 is shown the relationship in the saccharified-solution manufacturing method between the retention time of a substrate mixture and the saccharification rate in the saccharified-solution, when ammonia water having a concentration of 25% by mass is added to rice straw at a mass ratio of 1:1 and the yielded substrate mixture is heated. FIG. 4 shows the cases for heating temperatures of 80° C., 100° C., and 120° C., and FIG. 5 shows the cases for heating temperatures of 25° C., 50° C., 60° C., 80° C., and 100° C.

In FIG. 4, if the heating temperature is in the range of 80 to 120° C., by retention for 8 hours at respective temperatures, the saccharification rates is saturated, and there is little difference between the case of 100° C. and the case of 120° C. In FIG. 5, the saccharification rates saturated at 100 hours if the heating temperature is 25° C., at 1 hour if it is 100° C., and in the range of 1 to 100 hours depending on the respective temperatures, if it is in the range of 50 to 80° C.

Consequently, it is obvious that lignin can be dissociated adequately from the substrate, or the substrate can be swollen adequately, if the substrate mixture is retained at a temperature in the range of 25 to 100° C. for a time period in the range of 1 to 100 hours.

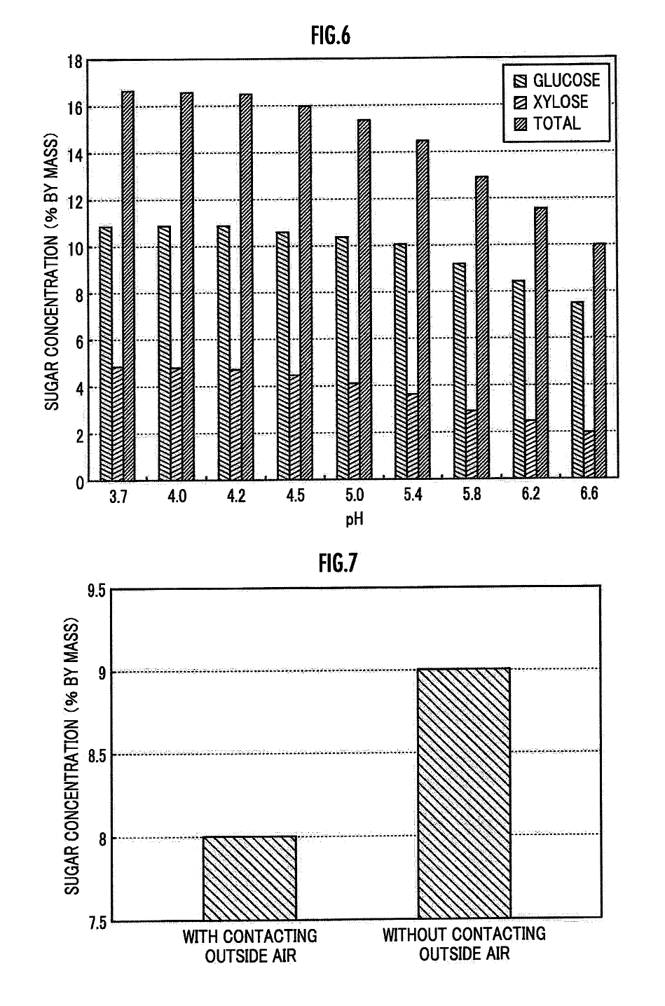

Next, in FIG. 6 is shown the relationship in the saccharified-solution manufacturing method between the pH of the substrate/saccharifying enzyme mixture liquid and the concentration of the yielded saccharified-solution, when the pH is varied in the range of 3 to 7. It is obvious from FIG. 6 that a saccharified-solution having a concentration in the range of 10 to 17% by mass can be yielded, if the pH of the substrate/saccharifying enzyme mixture liquid is set in the range of 3.70 to 6.55.

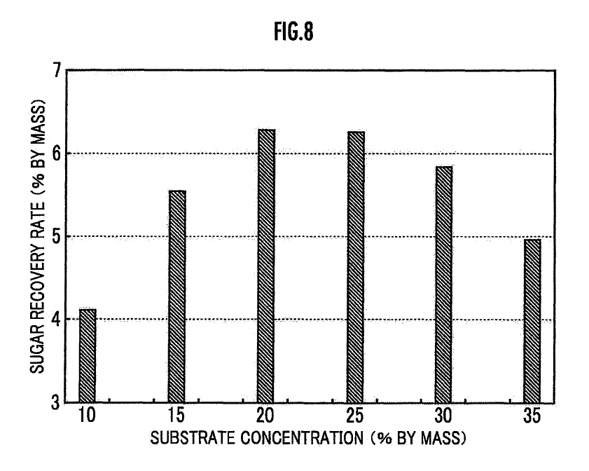

Next, in the saccharified-solution manufacturing method, the first saccharification treatment product yielded in the reaction vessel 2 was transferred to the enzymatic saccharification vessel 7 through the transfer line 6 without contacting the outside air and saccharified while being retained at a temperature of 40° C. for 144 hours in the enzymatic saccharification vessel 7. The concentration of the yielded saccharified-solution is shown in FIG. 7.

Further, in the saccharified-solution manufacturing method, the pretreated product for saccharification yielded in the reaction vessel 2 was transferred to the enzymatic saccharification vessel 7 without being converted to the first saccharification treatment product and under a condition allowing contact with the outside air, and was retained at a temperature of 40° C. for 144 hours for saccharification treatment in the enzymatic saccharification vessel 7. The concentration of the yielded saccharified-solution is shown in FIG. 7.

It is obvious from FIG. 7 that, if the first saccharification treatment product is transferred to the enzymatic saccharification vessel 7 without contacting the outside air and subjected to enzymatic saccharification treatment, a saccharified-solution with higher concentration can be yielded compared to a case where it is transferred under a condition allowing contact with the outside air.

Next, in the saccharified-solution manufacturing method, enzymatic saccharification treatment was carried out by adjusting the pH of the substrate/saccharifying enzyme mixture liquid to approx. 4, varying the content of a substrate in the range of 10 to 35% by mass with respect to the total mass of the substrate/saccharifying enzyme mixture liquid, and retaining the mixture at a temperature of 50° C. for 72 hours in the enzymatic saccharification vessel 7. After the saccharification treatment a yielded second saccharification treatment product was centrifuged (8000×g, 20 min) to separate and remove biomass residue and to yield a saccharified-solution.

The recovery rates of sugar yielded in the respective saccharified-solutions corresponding to the respective substrate contents with respect to the total mass of the substrate/saccharifying enzyme mixture liquid are shown in FIG. 8. It is obvious from FIG. 8 that by setting the substrate content in the range of 15 to 30% by mass with respect to the total mass of the substrate/saccharifying enzyme mixture liquid the recovery rate of the sugar can be higher and more sugar can be yielded as a saccharified-solution compared to the cases outside the above range.

### REFERENCE SIGNS LIST

1 . . . saccharified-solution manufacturing device, 2 . . . reaction vessel (first saccharification treatment unit), 3 . . . ammonia water tank (ammonia water supply unit), 4 . . . sulfuric acid tank (pH adjustment unit), 5 . . . enzyme tank (saccharifying enzyme addition unit), 6 . . . transfer line (transfer unit), 7 . . . enzymatic saccharification vessel (second saccharification treatment unit)

1. A saccharified-solution manufacturing method, in which lignocellulose-based biomass as a substrate is pretreated prior to saccharification to yield a pretreated product for saccharification in which lignin is dissociated from the substrate or the substrate is swollen, and thereafter the pretreated product for saccharification is saccharified enzymatically to yield a saccharified-solution, wherein the saccharified-solution manufacturing method comprises:
a step of mixing the substrate and ammonia water having a concentration in a range of 20 to 30% by mass at a mass ratio in a range of substrate:ammonia water=1:0.7 to 1:1.3 to yield a substrate mixture;
a step of heating and retaining the substrate mixture at a temperature in a range of 25 to 100° C. for a time period in a range of 1 to 100 hours to dissociate lignin from the substrate or to swell the substrate to yield a pretreated ammonia-containing product for saccharification;
a step of separating ammonia from the pretreated ammonia-containing product for saccharification through natural emission of ammonia gas to yield a substantially non-flowable pretreated product for saccharification;
a step of adding at least one acid of phosphoric acid, nitric acid and sulfuric acid to the pretreated product for saccharification after the ammonia separation to adjust a pH of the pretreated product for saccharification in a range of 3 to 7 and adding a saccharifying enzyme to yield a substrate/saccharifying enzyme mixture liquid containing the substrate in a range of 15 to 30% by mass with respect to the total mass;
a step of saccharifying enzymatically the substrate/saccharifying enzyme mixture liquid into a flowable state to yield a first saccharification treatment product;
a step of transferring the first saccharification treatment product to a next step under non-air-contact condition; and a step of saccharifying enzymatically the first saccharification treatment product to yield a saccharified-solution as a second saccharification treatment product.

2. (canceled)

3. The saccharified-solution manufacturing method according to claim 1, wherein the substrate/saccharifying enzyme mixture liquid is saccharified enzymatically into a flowable state using an enzyme for degrading cellulose and hemicellulose.

4. The saccharified-solution manufacturing method according to claim 1, wherein the first saccharification treatment product saccharified into a flowable state is saccharified enzymatically using an enzyme for degrading cellulose and hemicellulose.

5. The saccharified-solution manufacturing method according to claim 1, wherein the substrate/saccharifying enzyme mixture liquid is saccharified into a flowable state, which is transferred, when a viscosity of a first saccharification treatment product reaches a range of 30 to 1000 Pa·s, to the step of yielding a saccharified-solution.

6. A saccharified-solution manufacturing device, in which lignocellulose-based biomass as a substrate is pretreated prior to saccharification to yield to yield a pretreated product for saccharification in which lignin is dissociated from the substrate or the substrate is swollen, and thereafter the pretreated product for saccharification is saccharified enzymatically to yield a saccharified-solution, wherein the saccharified-solution manufacturing device comprises:
a first saccharification treatment unit configured:
to mix the substrate and ammonia water having a concentration in a range of 20 to 30% by mass at a mass ratio in a range of substrate:ammonia water=1:0.7 to 1:1.3 to yield a substrate mixture;
to heat and retain the yielded substrate mixture at a temperature in a range of 25 to 100° C. for a time period in a range of 1 to 100 hours to dissociate lignin from the substrate or to swell the substrate to yield a pretreated ammonia-containing product for saccharification;
to separate ammonia from the yielded pretreated ammonia-containing product for saccharification through natural emission of ammonia gas to yield a substantially non-flowable pretreated product for saccharification;
to adjust a pH of the pretreated product for saccharification after the ammonia separation in a range of 3 to 7 and add a saccharifying enzyme to yield a substrate/saccharifying enzyme mixture liquid containing the substrate in a range of 15 to 30% by mass with respect to the total mass; and
to saccharify enzymatically the substrate/saccharifying enzyme mixture liquid into a flowable state to yield a first saccharification treatment product;
an ammonia water supply unit configured to supply the ammonia water to the first saccharification treatment unit;
a pH adjustment unit configured to adjust the pH of the pretreated product for saccharification in the above range by adding at least one acid of phosphoric acid, nitric acid and sulfuric acid to the first saccharification treatment unit;
a saccharifying enzyme addition unit configured to add a saccharifying enzyme to the first saccharification treatment unit;
a transfer unit configured to transfer the first saccharification treatment product without contacting the outside air; and
a second saccharification treatment unit configured to perform an enzymatic saccharification treatment of the first saccharification treatment product transferred by the transfer unit to yield a saccharified-solution as a second saccharification treatment product.
